(19) United States
(12) Patent Application Publication
(10) Pub. No.: US 2015/0265506 A1
(43) Pub. Date: Sep. 24, 2015

(54) Foam-Promoting Compounds

(71) Applicant: CRODA AMERICAS LLC, Wilmington, DE (US)

(72) Inventor: Craig Bartlett Queen, Belmont, NC (US)

(21) Appl. No.: 14/734,589

(22) Filed: Jun. 9, 2015

Related U.S. Application Data
(63) Continuation of application No. 12/312,430, filed on Dec. 4, 2009.
(60) Provisional application No. 60/858,364, filed on Nov. 13, 2006.

(51) Int. Cl. A61K 8/04; A61Q 19/00; A61K 8/45

(52) U.S. Cl. CPC A61K 8/046; A61K 8/45; A61Q 19/00; A61K 2800/10

(57) ABSTRACT

The present invention relates to compounds according to Formula 1; wherein R represents a hydrocarbon group, Z represents an oxyethylene group, B represents an alkyl chain comprising 1 to 5 carbon atoms, Y represents 0 or 1, X represents a number greater than zero, and their use as surfactants, and in particular as surfactants which act as foam promoting agents.

### CROSS REFERENCE TO RELATED APPLICATION

This application claims the benefit of U.S. Provisional Patent Application No. 60/858,364, filed Nov. 13, 2006. The provisional application, in its entirety, is incorporated herein by reference.

### FIELD OF THE INVENTION

The present invention relates to novel compounds and their use as surfactants, in particular as surfactants which act as foam promoting agents.

### BACKGROUND OF THE INVENTION

Surfactants may be added to a cleansing system to alter the properties and/or esthetic qualities of the system, in particular to alter the rheology of the system. Rheology is the study of how materials deform and flow under the influence of external forces. Viscosity, which is the measure of resistance to flow, is one aspect of the scientific discipline of rheology.

The rheological properties of cosmetic preparations, such as shampoos, conditioners and skin treatment creams, are a key element of their acceptability in the marketplace. A consumer will purchase these products based on their perceived qualities. Even though a product may be otherwise functional, a consumer will often not repurchase a product, if the product fails to achieve the expected profile. Properties such as the rheology and sensory characteristics of the product are important elements which the consumer uses to judge the acceptability of the product.

Desirable properties of surfactants include the ability to increase viscosity (or thicken), provide foam boosting and foam stabilization and improve sensory characteristics of the composition to which they are added. Preferably surfactants should also increase the ability of a formulation to solubilise compounds such as fragrances.

Although non-ionic surfactants have been available for more than fifty years, only a limited number have a low enough melting point to be in the form of a readily flowable liquid form at room temperature. Non-ionic surfactants (such as cocamide monoethanolamide (MEA)) are typically solid at room temperature, and are heated during processing to melt the solid into a flowable form for subsequent incorporation into various formulations. However non-ionic surfactants which are liquid at room temperature are known. Commercially available PEG cocamides include Empilan MAA (ex Albright & Wilson UK Limited), Genagen CA-050 (ex Clariant Corporation), Hetoxamide C-4 (ex Globan Seven Inc.), Ninol 1301 (ex Stepan Company), Ninol C-5 (ex Stepan Company), Upamide C-5 (ex Universal Preserv-A-Chem, Inc).

Such heating is not only expensive, but may also affect other components of the resulting formulations. For example, incorporation of fragrances into melted surfactants may often result in the loss of the fragrances, as many of these substances are volatile oils. Furthermore, it is difficult to incorporate substances such as fragrances into conventional surfactants such as monoethanolamides due to the solid state of conventional surfactants at room temperature. Generally incorporating an oil-soluble substance such as a fragrance into an aqueous surfactant system involves initially mixing the oil-soluble substance with a liquid surfactant first. This additional processing step decreases the yield of processing and increases the cost and complexity of processing.

Typically, liquid alkyl diethanolamide (DEA) surfactants have been used as foam boosters and thickeners in liquid cleansing systems. One of the most favourable attributes of diethanolamides is their liquidity at room temperature. This allows cleansing products to be manufactured without the additional step of heating the production batch thereby saving the cost of providing the heat energy needed. However, diethanolamides have unfavourable characteristics and are frequently associated with diethanolamines, which can react with nitrogen oxides and sodium nitrite to form nitrosamines, which are known to be carcinogenic.

Consequently, it is widely acknowledged that diethanolamides are not suitable for inclusion in future surfactant formulations due to possible regulatory and consumer preference considerations.

Individual propoxylated alkanolamide surfactants for cleansing systems and methods of preparation have been disclosed in U.S. Pat. No. 6,531,443-B. These modified alkanolamides may be derived from capryl, stearic, soy oil, and coconut oil fatty monoethanolamides. While each of these materials has many useful properties, they may have potential drawbacks if used individually as the sole thickener. Certain properties of these materials which may be improved include one or more of the following: colour stability, viscosity increasing performance, or foam boosting performance as well as compatibility with some surfactant systems. One example is polypropylene glycol (PPG) hydroxyethyl caprylamide, which provides excellent colour stability and is a good foam booster, but has little viscosity building character. Another example is PPG hydroxyethyl cocamide, which is compatible with nearly all surfactant systems and has good colour stability, but does not build viscosity well in comparison to cocamide MEA, from which it is derived. A third example, PPG hydroxyethyl soyamide (a straight chained unsaturated C-I8 derived from soy bean oil) provides viscosity building character, but not colour stability.

U.S. Pat. No. 6,531,443-B discloses the addition of modified alkanolamides as surfactants to increase the viscosity of personal care compositions, especially compositions for hair care and skin treatment. However, the addition of such alkanolamides does not enhance the foaming properties of the composition to which they are added. Typically the addition of such alkanolamides may have a deleterious effect on the foaming properties of the composition. In particular, the addition of such alkanolamides may inhibit foam formation, and reduce the sustainability of any foam formed.

### SUMMARY OF THE INVENTION

We have now surprisingly discovered certain non-ionic surfactants which reduce or substantially overcome at least one of the aforementioned problems.

According to a first aspect of the present invention there is provided a compound according to Formula 1

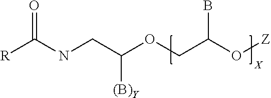

wherein:
R represents a hydrocarbon group,
Z represents an oxyethylene group,
B represents an alkyl chain comprising 1 to 5 carbon atoms,
Y represents 0 or 1; and
X represents a number greater than zero.

Suitably R represents a hydrocarbon group comprising 1 to 25 carbon atoms.

Suitably Z comprises 1 to 12 mole, more suitably 2 to 10 mole, and advantageously 3 to 8 mole of oxyethylene.

Y is preferably 0.

Typically B represents CH₃ or CH₂—CH₃, and is preferably CH₃.

In one embodiment X is from 1 to 6.

The compounds of Formula 1 are useful as surfactants. The compounds of Formula 1 also act as foam promoting agents, typically increasing the amount of foaming of a substance and the sustainability of foam formed. Typically the compounds of Formula 1 are as effective at promoting foaming characteristics as known foam promoting agents such as PEG 5 cocamide. This is particularly surprising as alkanolamides such as those disclosed in U.S. Pat. No. 6,531,443-B reduce the ability of a substance to foam. Typically the compounds of Formula 1 have good associated colour stability.

The compounds of Formula 1 typically have a congealing temperature less than about 25° C., and are preferably liquids at ambient temperature or lower (e.g. 25° C. or lower). As such, the compounds of the present invention may generally be processed without heating. Typically the compounds of Formula 1 may be added to a substance in order to enhance the foaming properties of the substance without heating. Accordingly the cost and inconvenience of processing is reduced compared to known non-ionic surfactants which are generally solid at room temperature. Due to the relatively low melting point of the compounds of the present invention, formulations comprising these compounds may be cold processed avoiding the risk of damaging other components of the resulting formulations. As such, heat sensitive components, such as oil-soluble components (in particular perfumes) may be added without risk of damage.

The cost and complexity of producing compositions comprising the compounds of Formula 1 is minimised accordingly. The relatively low melting points of the compounds of the present invention provide an important advantage over conventional foam boosting compounds such as PEG 5 Cocamide.

Formulations comprising the compounds of the present invention have surprisingly good sensory characteristics. The formulations have good associated “wet feel” and “dry feel” characteristics. Generally the formulations comprising the compounds of the present invention have better associated sensory characteristics than corresponding formulations comprising known surfactants such as the modified alkanolamides of U.S. Pat. No. 6,531,443-B or known foam boosting agents such as PEG 5 Cocamide.

Suitably the oxyethylene group has the structure:

—[—CH₂—CH₂—O—]ₓ—H

wherein x=1 to 12.

1 mole of compound according to Formula 1 may comprise 1 to 12 mole of oxyethylene group, suitably 2 to 10 mole of oxyethylene group, more suitably 3 to 8 mole of oxyethylene group; yet more suitably 3, 6 or 8 mole of oxyethylene group; advantageously 6 mole of oxyethylene group.

R may represent an optionally substituted, saturated or unsaturated hydrocarbon group wherein the carbon backbone of the hydrocarbon group may comprise 1 to 25 carbon atoms, suitably 3 to 21 carbon atoms; more suitably 8 to 18 carbon atoms.

X may represent an integer of 1 to 10; suitably an integer of 1 to 6, more suitably an integer of 1 to 4. Preferably X represents 1, 2 or 3.

The carbon backbone of the compound according to Formula 1 may comprise further substitutions than those shown in Formula 1.

According to a further aspect of the present invention there is provided a composition comprising one or more compounds according to Formula 1 and one or more excipients.

The amount of compound of Formula 1 present in the composition of the present invention depends on the type of composition. Typically the composition comprises about 0.1 to 50% by weight compound of Formula 1; suitably about 0.1 to 10% by weight; more suitably about 1 to 5% by weight; advantageously about 3% by weight.

Suitably the composition is a cosmetic preparation; in particular a personal care preparation such as a cosmetic preparation for hair care or skin care. In this connection, typical personal care formulations include shampoos, hair colorants, hair conditioners, bath products and skin treatment creams and lotions, including skin treatment creams and lotions for babies, body washes including facial washes and wipes such as cleansing wipes, in particular for use in connection with babies or small children; including skin treatment creams and lotions for babies. Other formulations where the properties of the novel compositions of Formula 1 can be utilized include make-up creams, sunscreens, skin-toners, antiperspirants and the like.

Alternatively the composition of the present invention may be a home care or an industrial formulation such as light duty detergents, laundry detergents, hard surface cleaners, industrial cleaners, metal working and lubricating agents, emulsifiers, anti-corrosion agents for metal products and various other liquid and/or water based personal care, home care, industrial, crop and textile compositions.

Suitably the composition may comprise excipients, such as fillers, solvents, surfactants and/or stabilisers. The composition may comprise conventional additives such as perfumes, preservatives, complexing agents, opacifiers, luster developing agents and the like, oils, emollients and suspended particles (e.g. exfoliating or oil-containing beads). Typically the composition comprises anionic, non-ionic, amphoteric and/or cationic surfactants. Suitably the composition comprises sodium lauryl sulfate (SLS), sodium laureth sulfate (SLES) and/or a betaine.

The compounds of the present invention typically boost the foaming properties of the composition of the present invention relative to corresponding compositions comprising known surfactants or viscosity increasing components.

In particular, the amount of foaming generated by the composition of the present invention is typically increased relative to compositions comprising known surfactants or known viscosity increasing components, and the sustainability of the foam generated is generally increased. The foam generated by the composition of the present invention is typically denser and/or covers a greater area than the foam generated by compositions which do not comprise the compounds of the present invention.

According to a further aspect of the present invention there is provided a method of enhancing the foaming properties or providing improved sensorial delivery of a substance comprising the step of adding one or more compounds according to Formula 1.

The term “substance” refers to the composition or formulation to which the compounds of the present invention may be added. The foaming properties of the substance are enhanced and the sensorial delivery of the substance is increased following addition of the compounds of the present invention.

According to one embodiment the amount and density of the foam generated by the substance is increased through the addition of one or more compounds according to Formula 1. Typically the amount of foam generated by the substance is increased by about at least 10%; suitably by about at least 20%; more suitably by about at least 50%. Typically the density of the foam generated is increased by about at least 10%; suitably by about at least 20%; more suitably by about at least 50%.

Advantageously the method of the present invention increases the sustainability of the foam generated by the substance. Typically the foam generated is sustainable for about at least 10% longer; more suitably about at least 20% longer; preferably about at least 50% longer.

Generally the composition formed through the addition of one or more compounds according to Formula 1 to the substance comprises about 0.1 to 50% by weight compound of Formula 1; suitably about 0.1 to 10% by weight; more suitably about 1 to 5% by weight; advantageously about 3% by weight.

As noted above the compounds of the present invention generally have a congealing temperature less than about 25° C. and are preferably liquid at ambient temperatures or lower (e.g. 25° C. or lower). The method of enhancing the foaming properties of a substance does not generally require elevated temperatures to proceed. The compounds of the present invention may be added to the substance at non-elevated temperatures. The compounds of Formula 1 may be added to the substance at temperatures less than 80° C., suitably the compounds of Formula 1 are added to the substance at room temperature (i.e. 20 to 25° C.). The method may include a mixing step following addition of the compound(s) of Formula 1.

As noted above, the addition of the compounds of Formula 1 to a substance also typically improve the sensory characteristics of the substance relative to known viscosity increasing agents such as the alkanolamides disclosed in U.S. Pat. No. 5,531,443-B, or relative to known foam boosting agents such as PEG 5 Cocamide.

According to a further aspect of the present invention there is provided a method of forming a compound according to Formula 1 comprising the steps of contacting a compound according to Formula 2 with ethylene oxide:

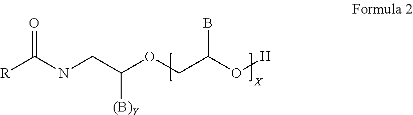

Groups R, B, Y and X are as defined above.

Examples of useful compounds of Formula 2 include polyoxypropylene-, polyoxybutylene-, fatty ethanolamides or fatty isopropanolamides. Propoxylated fatty ethanolamides are preferred. The fatty ethanolamide moiety is preferably a fatty monoethanolamide, and more preferably is derived from lauric monoethanolamide, capric monoethanolamide, caprylic monoethanolamide, caprylic/capric monoethanolamide, decanoic monoethanolamide, myristic monoethanolamide, palmitic monoethanolamide, stearic monoethanolamide, isostearic monoethanolamide, oleic monoethanolamide, linoleic monoethanolamide, octyldecanoic monoethanolamide, 2-heptylundecanoic monoethanolamide, coconut oil fatty monoethanolamide, beef tallow fatty monoethanolamide, soy oil fatty monoethanolamide and palm kernel oil fatty monoethanolamide. Of these capryl, linoleyl, stearic, isostearic, soy oil, and coconut oil fatty monoethanolamides are preferred. When isostearic is used it is preferably used in combination with another of the fatty alkanolamides.

A particularly preferred compound of Formula 2 is selected from the group consisting of PPG-1 hydroxyethyl caprylamide, PPG-2 hydroxyethyl cocamide, PPG-2 hydroxyethyl isostearamide, and mixtures thereof.

In an alternative embodiment, propoxylated fatty isopropanolamides are employed as compounds of Formula 2. The fatty isopropanolamide moiety is preferably a fatty monoisopropanolamide, and more preferably is derived from lauric monoisopropanolamide, capric monoisopropanolamide, caprylic monoisopropanolamide, caprylic/capric monoisopropanolamide, decanoic monoisopropanolamide, myristic monoisopropanolamide, palmitic monoisopropanolamide, stearic monoisopropanolamide, isostearic monoisopropanolamide, oleic monoisopropanolamide, linoleic monoisopropanolamide, octyldecanoic monoisopropanolamide, 2-heptylundecanoic monoisopropanolamide, coconut oil fatty monoisopropanolamide, beef tallow fatty monoisopropanolamide, soy oil fatty monoisopropanolamide, and palm kernel oil fatty monoisopropanolamide. Of these, stearic, isostearic, and coconut oil fatty monoisopropanolamide are preferred.

Each mole of compound according to Formula 2 may be contacted with 1 to 12 moles of ethylene oxide: suitably with 2 to 10 moles of ethylene oxide; more suitably 3 to 8 moles of ethylene oxide; advantageously around 6 moles of ethylene oxide.

The method may include the addition of a catalyst, such as sodium methoxide solution.

The method of forming the compounds of the present invention may proceed at elevated temperatures; for instance at temperatures of 100° C. or more; suitably 150° C. or more. Typically the method of the present invention proceeds at temperatures of around 160° C. A pressure of up to about 10 barg (12 bara); suitably 5 barg (6 bara) may be applied.

The method of the present invention may proceed for up to about 24 hours or more.

The present invention will now be described by way of example only.

PPG-1 hydroxyethyl caprylamide (Promidium™ CC, ex Croda), PPG-2 hydroxyethyl cocamide (Promidium™ CO, ex Croda), and PPG-2 hydroxyethyl isostearamide (Promidium™ IS, ex Croda) are conventionally used as viscosity increasing agents.

Embodiments of the invention will now be described, by way of example only, which are non-limiting to the scope of the invention.

### EXAMPLE 1

### Preparation of Ethoxylated PPG-1 Hydroxyethyl Caprylamide

491.4 g of PPG-1 hydroxyethyl caprylamide (Promidium™ CC, ex Croda) were charged to the reactor with 4.9 g of 30% sodium methoxide solution as catalyst, and dehydrated to remove methanol. 491.4 g of this mixture was then reacted with 233 g of ethylene oxide at 160° C. to give nominally 3 moles EO per PPG-1 hydroxyethyl caprylamide unit. 529.3 g of the resultant ethoxylated product was further reacted with 170 g of ethylene oxide at 160° C. to give a total of 6 moles EO per PPG-1 hydroxyethyl caprylamide unit. Finally, 496.4 g of the resultant ethoxylated product was then reacted with 81 g of ethylene oxide to give 8 moles EO per PPG-1 hydroxyethyl caprylamide unit.

The resultant ethoxylated PPG-1 hydroxyethyl caprylamide compounds ethoxylated with 3, 6 and 8 mole ethylene oxide were all liquid at room temperature.

### EXAMPLE 2

### Preparation of Ethoxylated PPG-2 Hydroxyethyl Cocamide

454.9 g of PPG-2 hydroxyethyl cocamide (Promidium™ CO, ex Croda) were charged to the reactor with 3.03 g of 30% sodium methoxide solution as catalyst, and dehydrated to water content 0.01% w/w.

388.2 g of this mixture was then reacted with 271 g of ethylene oxide at 160° C. to give 3 moles EO per PPG-2 hydroxyethyl cocamide unit. 535 g of the resultant ethoxylated product was further reacted with 220 g of ethylene oxide at 160° C. to give a total of 6 moles EO per PPG-2 hydroxyethyl cocamide unit. Finally, 607.2 g of the resultant ethoxylated product was then reacted with 118 g of ethylene oxide to give 8 moles EO per PPG-2 hydroxyethyl cocamide unit.

| | 1% aqueous cloudpoint |
|---|---|
| Analysis | (° C.) |
| PPG-2 hydroxyethyl cocamide + 3 mole EO | 46.2 |
| PPG-2 hydroxyethyl cocamide + 6 mole EO | 75.5 |
| PPG-2 hydroxyethyl cocamide + 8 mole EO | 86.2 |

### EXAMPLE 3

### Preparation of Ethoxylated PPG-2 Hydroxyethyl Isostearamide

496.8 g of PPG-2 hydroxyethyl isostearamide (Promidium™ IS, ex Croda) were charged to the reactor with 3.3 g of 30% sodium methoxide solution as catalyst, and dehydrated to remove methanol. 496.8 g of this mixture was then reacted with 226 g of ethylene oxide at 160° C. to give 3 moles EO per PPG-2 hydroxyethyl isostearamide unit. 562.9 g of the resultant ethoxylated product was further reacted with 176 g of ethylene oxide at 160° C. to give a total of 6 moles EO per PPG-2 hydroxyethyl isostearamide unit. Finally, 542.2 g of the resultant ethoxylated product was then reacted with 86 g of ethylene oxide to give 8 moles EO per PPG-2 hydroxyethyl isostearamide unit.

### EXAMPLE 4

### Comparison of Physical Properties

PPG-1 hydroxyethyl caprylamide and PPG-2 hydroxyethyl cocamide were ethoxylated with 3, 6 and 8 moles of ethylene oxide as described above. The physical properties of the resultant ethoxylated compounds were tested, and compared to the physical properties associated with PPG-1 hydroxyethyl caprylamide and PPG-2 hydroxyethyl cocamide. The physical properties of the ethoxylated compounds were also compared to PEG 5 Cocamide which is conventionally used as a foam boosting agent. In particular the foaming properties and the sensory characteristics of the compounds were compared. Testing was done on solutions comprising the resultant ethoxylated compounds in an SLS/SLES/betaine base wherein the solutions comprise 3.0% of the resultant ethoxylated compounds or 3.0% PPG-1 hydroxyethyl caprylamide, PPG-2 hydroxyethyl cocamide or PEG 5 Cocamide compound. The results of the comparison are summarised below.

### Preliminary Foam & Skin Feel Panel Results

Ross Miles Foam

| | Initial Avg. Height | 5 Min Avg. Height |
|---|---|---|
| | (mm) | (mm) |
| PPG-1 hydroxyethyl | 67.5 | 60 |
| caprylamide | | |
| PPG-1 hydroxyethyl | 145 | 135 |
| caprylamide + 6 mole EO | | |
| PPG-2 hydroxyethyl | 125 | 120 |
| cocamide | | |
| PPG-2 hydroxyethyl | 137.5 | 137 |
| cocamide + 6 mole EO | | |
| GENAGEN CA 050 | 137.5 | 137 |
| (PEG 5 Cocamide) | | |

Panel Testing (6 Panelists then 10 Panelists for Final Screen)

Hand Wash

| | PPG-2 hydroxyethyl | | PPG-2 hydroxyethyl | PEG 5 |
|---|---|---|---|---|
| | cocamide + 6 EO | | cocamide + 6 EO | Cocamide |
| Most Foam | 2 | 4 | 8 | 1 (1=) |
| Most Dense | 2 | 3 (1=) | 6 | 2 (2=) |
| Best Wet Feel | 0 | 5 (1=) | 4 | 3 (3=) |
| Best Dry Feel | 0 | 6 | 6 | 2 (2=) |

(Note:
“(1=)” means that one panel member did not distinguish a difference)

The data above evidences the improved foaming properties of the compounds of the present invention compared to conventional viscosity increasing agents. Furthermore, the compounds of the present invention provide better associated sensory characteristics than conventional viscosity increasing.

The compounds of the present invention provide enhanced foaming properties and enhanced sensory characteristics. This combination of characteristics means that the compounds of the present invention are extremely useful additives for preparations, in particular cosmetic preparations.

All documents referred to in this specification are hereby incorporated by reference. Various modifications and variations to the described embodiments of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1-27. (canceled)

28. A method of enhancing foaming properties and/or providing improved sensorial delivery of a personal care formulation, comprising:
adding to the personal care formulation a foam-promoting compound represented by Formula 1: 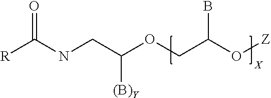
wherein:
R represents a saturated hydrocarbon group wherein the carbon backbone of the hydrocarbon group comprises 3 to 21 carbon atoms;
Z represents 3 to 8 moles of an oxyethylene group;
B represents —CH₃ or —CH₂CH₃;
Y represents 0 or 1; and
X represents an integer of 1 or 2.

29. The method of claim 28, wherein the personal care formulation is a shampoo, hair colorant, hair conditioner, bath product, body wash, facial wash, skin treatment cream, skin treatment lotion, make-up cream, sunscreen, skin-toner, or antiperspirant.

30. The method of claim 28, wherein the personal care formulation further comprises one or more of fillers, solvents, surfactants, stabilisers, perfumes, preservatives, complexing agents, opacifiers and luster developing agents, oils, emollients, suspended particles, anionics, non-ionics, amphoterics and cationics.

31. The method of claim 28, wherein the foam-promoting compound represented by Formula 1 is added to the personal care formulation at a temperature of 20 to 25° C.

32. The method of claim 28, wherein the addition of the foam-promoting compound represented by Formula 1, without heating, enhances the foaming properties of the personal care formulation, comprising:
a) an increase in the amount of foam generated by the personal care formulation;
b) an increase in the density of foam generated by the personal care formulation;
c) an increase in the sustainability of the foam generated by the personal care formulation; or
d) an increase in the area covered by the foam generated by the personal care formulation;
relative to a personal care formulation exclusive of the foam-promoting compound represented by Formula 1.

33. The method of claim 32, wherein the increased amount of the foam generated by the personal care formulation is at least 10%.

34. The method of claim 32, wherein the increased amount of the foam generated by the personal care formulation is at least 20%.

35. The method of claim 32, wherein the increased density of the foam generated by the personal care formulation is at least 10%.

36. The method of claim 32, wherein the increased density of the foam generated by the personal care formulation is at least 20%.

37. The method of claim 32, wherein the increased sustainability of the foam generated by the personal care formulation is at least 10% longer.

38. The method of claim 32, wherein the increased sustainability of the foam generated by the personal care formulation is at least 20% longer.

39. The method of claim 28, wherein the carbon backbone of the hydrocarbon group comprises 8 to 18 carbon atoms.

40. The method of claim 28, wherein the carbon backbone of the hydrocarbon group is unsubstituted.

41. The method of claim 28, wherein the carbon backbone of the hydrocarbon group is substituted.

42. The method of claim 28, wherein Y=0.

43. The method of claim 28, wherein Y=1.

44. The method of claim 28, wherein Z represents 3 moles of the oxyethylene group.

45. The method of claim 28, wherein Z represents 6 moles of the oxyethylene group.

46. The method of claim 28, wherein Z represents 8 moles of the oxyethylene group.

47. The method of claim 28, wherein the foam-promoting compound represented by Formula 1 is:
3-ethylene glycol PPG-1 hydroxyethyl caprylamide, 6-ethylene glycol PPG-1 hydroxyethyl caprylamide, 8-ethylene glycol PPG-1 hydroxyethyl caprylamide, 3-ethylene glycol PPG-2 hydroxyethyl cocamide, 6-ethylene glycol PPG-2 hydroxyethyl cocamide, 8-ethylene glycol PPG-2 hydroxyethyl cocamide, 3-ethylene glycol PPG-2 hydroxyethyl isostearamide, 6-ethylene glycol PPG-2 hydroxyethyl isostearamide, or 8-ethylene glycol PPG-2 hydroxyethyl isostearamide.

48. The method of claim 28, wherein the foam-promoting compound represented by Formula 1 has a congealing temperature of less than about 25° C.

49. The method of claim 28, wherein the foam-promoting compound represented by Formula 1 is added as a composition to the personal care formulation.

50. The method of claim 49, wherein the added composition further comprises one or more excipients.

51. The method of claim 49, wherein the composition comprises 1 to 5% by weight of the foam-promoting compound represented by Formula 1.
